(19) United States
(12) Patent Application Publication
(10) Pub. No.: US 2009/0270820 A1
(43) Pub. Date: Oct. 29, 2009

(54) SYSTEM AND METHOD FOR USE OF AGENT IN COMBINATION WITH SUBATMOSPHERIC PRESSURE TISSUE TREATMENT

(76) Inventors: Royce W. Johnson, Universal City, TX (US); Martin W. Steiner, Fletcher, NC (US); Devin C. Ginther, Dripping Springs, TX (US); Archel Ambrosio, San Antonio, TX (US); Teryl Blane Sanders, San Antonio, TX (US)

Correspondence Address: KINETIC CONCEPTS, INC.;C/O SONNENSCHEIN NATH & ROSENTHAL LLP, P.O. BOX 061080, WACKER DRIVE STATION, WILLIS TOWER, CHICAGO, IL, 60606, US

(21) Appl. No.: 12/431,667

(22) Filed: Apr. 28, 2009

Related U.S. Application Data
(63) Continuation of application No. 11/497,457, filed on Aug. 1, 2006, now abandoned.
Application No. 11/497,457 is a continuation-in-part of application No. 09/937,942, filed on Oct. 2, 2001, now Pat. No. 7,534,240.
Application No. 09/937,942 is a continuation-in-part of application No. 11/494,171, filed on Jul. 26, 2006, now abandoned.
Application No. 11/494,171 is a continuation-in-part of application No. 11/189,195, filed on Jul. 26, 2005.
(60) Provisional application No. 60/127,595, filed on Apr. 2, 1999.
(60) Provisional application No. 60/591,014, filed on Jul. 26, 2004.

(51) Int. Cl. A61L 15/42; A61F 13/00; A61M 1/00

(52) U.S. Cl. 604/290; 604/313; 604/304

(57) ABSTRACT

A reduced pressure therapy system includes a foam pad having a plurality of passages to distribute a reduced pressure to a tissue. The foam pad includes exterior surfaces and interior surfaces along the plurality of passages. A silver coating uniformly covers the exterior surfaces of the foam pad and the interior surfaces along the passages. A drape is adapted to be positioned over the foam pad to maintain a sealable space over the wound, and a vacuum source is in fluid communication with the sealable space to deliver the reduced pressure to the sealable space.

### CROSS-REFERENCE TO RELATED APPLICATIONS

This application is a continuation of U.S. patent application Ser. No. 11/497,457, filed Aug. 1, 2006, which is continuation-in-part of U.S. patent application Ser. No. 09/937,942, filed Oct. 2, 2001, which is a national stage application of International Application No. PCT/US00/08821, filed Mar. 31, 2000, which claims the benefit of U.S. Provisional Application No. 60/127,595, filed Apr. 2, 1999; this application is a continuation-in-part of U.S. patent application Ser. No. 11/494,171, filed Jul. 26, 2006, which is a continuation-in-part of U.S. patent application Ser. No. 11/189,195, filed Jul. 26, 2005, which claims the benefit of U.S. Provisional Application No. 60/591,014, filed Jul. 26, 2004. All of the above-referenced applications are hereby incorporated by reference.

### BACKGROUND OF THE INVENTION

1. Field of the Invention

This invention relates to the healing of wounds and other tissue. More specifically, but not by way of limitation, it relates to the subatmospheric pressure therapy of wounds, commercialized by KCI USA, Inc. of San Antonio, Tex., in the form of its “VACUUM ASSISTED CLOSURE®” (or “V.A.C.®”) subatmospheric pressure tissue treatment product line, and wherein a growth factor or other agent is introduced to a wound or tissue site through grafting with a pad in order to facilitate healing.

2. Description of Related Art

Subatmospheric pressure-induced healing of tissue, including but not limited to wounds, has been commercialized by KCI USA, Inc. of San Antonio, Tex., in the form of its “Vacuum Assisted Closures® or “V.A.C.®” subatmospheric pressure therapy product line. The subatmospheric pressure-induced healing process in epithelial and subcutaneous tissues was first described in U.S. Pat. Nos. 5,636,643 and 5,645,081 issued to Argenta et al., on Jun. 10, 1997 and Jul. 8, 1997 respectively, the disclosures of which are incorporated by reference as though fully set forth herein. A dressing that was later found to be useful for subatmospheric pressure-induced healing has also been described in commonly assigned U.S. Pat. No. 4,969,880 issued on Nov. 13, 1990 to Zamierowski, as well as its continuations and continuations in part, U.S. Pat. No. 5,100,396, issued on Mar. 31, 1992, U.S. Pat. No. 5,261,893, issued Nov. 16, 1993, and U.S. Pat. No. 5,527,293, issued Jun. 18, 1996, the disclosures of which are incorporated herein by this reference. Further improvements and modifications of such a dressing are also described in U.S. Pat. No. 6,071,267, issued on Jun. 6, 2000 to Zamierowski. Additional improvements have also been described in U.S. Pat. No. 6,142,982, issued on May 13, 1998 to Hunt, et al., and U.S. Pat. No. 7,004,915 issued on Feb. 28, 2006 to Boynton, et al., the disclosures of which are incorporated by reference as though fully set forth herein. Improvements in the use and operation of the connection and conduit components between the dressing and the source of subatmospheric pressure instrumentation have been described in the U.S. provisional patent application Ser. No. 60/765,548, entitled SYSTEMS AND METHODS FOR IMPROVED CONNECTION TO WOUND DRESSINGS IN CONJUNCTION WITH REDUCED PRESSURE WOUND TREATMENT SYSTEMS filed Feb. 6, 2006, the disclosure of which is incorporated by reference as though fully set forth herein. A process for uniformly covering the dressing with antimicrobial agents, and an improved dressing formed by the process, and a subatmospheric pressure wound therapy system and dressing with antimicrobial effects has been described in commonly assigned U.S. patent application Ser. No. 11/189,195 filed Jul. 26, 2005, and its copending continuation-in-part U.S. patent application entitled SYSTEM AND METHOD FOR USE OF AGENT IN COMBINATION WITH SUBATMOSPHERIC TISSUE TREATMENT, filed Jul. 26, 2006, the disclosures of which are incorporated herein by this reference.

Wound closure involves the inward migration of epithelial and subcutaneous tissue adjacent the wound. This migration is ordinarily assisted through the inflammatory process, whereby blood flow is increased and various functional cell types are activated. Through the inflammatory process, blood flow through damaged or broken vessels is stopped by capillary level occlusion, whereafter cleanup and rebuilding operations may begin. Unfortunately, this process is hampered when a wound is large or has become infected. In such wounds, a zone of stasis (i.e. an area in which localized swelling of tissue restricts the flow of blood to the tissues) forms near the surface of the wound.

Without sufficient blood flow, the epithelial and subcutaneous tissues surrounding the wound not only receive diminished oxygen and nutrients, but are also less able to successfully fight bacterial infection and thus are less able to naturally close the wound. Until recently, such difficult wounds were addressed only through the use of sutures or staples. Although still widely practiced and often effective, such mechanical closure techniques suffer a major disadvantage in that they produce tension on the skin tissue adjacent the wound. In particular, the tensile force required in order to achieve closure using sutures or staples causes very high localized stresses at the suture or staple insertion point. These stresses commonly result in the rupture of the tissue at the insertion points, which can eventually cause wound dehiscence and additional tissue loss.

Additionally, some wounds harden and inflame to such a degree due to infection that closure by stapling or suturing is not feasible. Wounds not reparable by suturing or stapling generally require prolonged hospitalization, with its attendant high cost, and major surgical procedures, such as grafts of surrounding tissues. Examples of wounds not readily treatable with staples or suturing include large, deep, open wounds; decubitus ulcers; ulcers resulting from chronic osteomyelitis; and partial thickness burns that subsequently develop into full thickness burns.

As a result of these and other shortcomings of mechanical closure devices, methods and apparatus for healing wounds by applying continuous subatmospheric pressures have been developed. When applied over a sufficient area of the wound, such subatmospheric pressures have been found to promote healing. In practice, the application to a wound of subatmospheric pressure, commonly referred to as subatmospheric pressure tissue treatment, or subatmospheric pressure wound therapy, or negative pressure wound therapy (NPWT) and commercialized as Vacuum Assisted Closure®, or V.A.C.®, by KCI USA, Inc. of San Antonio, Tex., typically involves mechanical contraction of the wound with simultaneous removal of excess and interstitial body-liquid. In this manner, subatmospheric pressure therapy cooperates with the body's natural inflammatory process while alleviating many of the known intrinsic side effects, such as edema caused by increased blood flow absent the necessary vascular structure for proper removal of waste liquids.

While subatmospheric pressure wound therapy has been highly successful in the promotion of wound closure, healing many wounds previously thought largely untreatable, some difficulty remains. Because the inflammatory process is very unique to the individual patient, even the addition of subatmospheric pressure wound therapy does not result in a fast enough response, especially during the occlusion and initial cleanup and rebuilding stages, for adequate healing of some wounds. It is therefore a principle object of the following embodiments to provide a method and apparatus whereby the known subatmospheric pressure wound therapy modalities are improved through the introduction of growth factors and/or other agents that facilitate wound healing.

### SUMMARY

These and other needs are met through a method and apparatus for the introduction of a wound healing agent to a tissue site, such as a wound, undergoing subatmospheric pressure wound therapy, generally comprising a foam pad for insertion substantially into a wound site; and a wound drape for sealing enclosure of the foam pad at the wound site. The foam pad is placed in fluid communication with a source of subatmospheric pressure for promotion of wound healing. Additionally, the foam pad is predisposed, through grafting or other techniques known to those of ordinary skill in the art, with basic fibroblast growth factor (bFGF), anti-microbials or other factors, also known to those of ordinary skill in the art, for the promotion of increased wound healing.

The foam or dressing predisposed with healing factors bound to the surface of the foam or dressing material serves as a screen for use in delivering the healing factors, such as growth factors and antimicrobial agents, to the tissue site in combination with the application of a subatmospheric pressure. The foam or dressing serves as a substrate to which the healing factors may be bound using the coating processes discussed herein, or other techniques known to those of ordinary skill in the art.

According to one embodiment, a growth factor or other wound healing agent is added to the previously known subatmospheric pressure therapy through modification as necessary of the foam pad or dressing material. Such growth factors as the basic fibroblast growth factor (bFGF) are known to accelerate wound healing due to their potent angiogenesis and granulation tissue formation activities. As has been demonstrated even with difficult to heal wounds, such as infected wounds, burn wounds, and diabetic wounds, the resultant activities lead to the rapid reepithelialization and contraction of the wound. The combination of subatmospheric pressure therapy with growth factor introduction, through the modification of the foam pad and predisposition thereof with the bFGF, is therefore thought to be an important contribution to the wound healing arts.

According to one preferred embodiment, an antimicrobial agent, such as silver, is added to the previously known subatmospheric pressure therapy through modification as necessary of the foam pad or dressing. Specifically, a process for uniformly coating the foam pad or other dressing, and a foam or dressing formed by this process with a polymer-based coating or a metal-based coating; its use with a subatmospheric pressure tissue treatment device; and a subatmospheric pressure tissue treatment system and dressing with antimicrobial effects is disclosed.

In practice, the screen is placed in contact with tissue and a cover is positioned to enclose the screen. The cover also serves to define a space between the cover and the tissue. A pathway is provided between the source of subatmospheric pressure and the space defined by the cover, for application of the subatmospheric pressure within the space defined by the cover. A container is connected to the pathway between the source of subatmospheric pressure and the cover. The container receives the body-liquid drawn along the pathway from within the space defined by the cover.

At least a portion of the screen is the substrate predisposed, through a polymer-based or metal-based coating process, with a uniform covering of a coating comprising at least one therapeutic or prophylactic agents. The coating releases at least a portion of the agents within the space defined by the cover. The exterior and interior surfaces of the substrate are covered with the coating to enable the user to expose at least one coated surface of the uniformly covered substrate portion of the screen when adjusting the size and shape of the screen to fit the tissue site.

During application of the subatmospheric pressure within the space defined by the cover, an area of contact between the tissue and the uniformly covered substrate portion of the screen is increased as the tissue microdeforms and the screen compresses and conforms to the surface of the tissue. The coating releases at least a portion of the agents directly to the area of contacted tissue.

In another embodiment, a process for adapting the substrate for treating the tissue during application of the subatmospheric pressure tissue treatment includes the steps of creating a coating solution comprising at least one therapeutic or prophylactic agent; uniformly coating the substrate with the coating comprising the agents, such that an upper surface, a lower surface, side surfaces, and interior surfaces of the screen are uniformly coated; and severing the uniformly coated screen to match the size and shape of the tissue site, such that all exposed surfaces of the screen are uniformly coated sufficient to treat the tissue site during application of the subatmospheric pressure.

The process may further include steps for positioning the screen in contact with the tissue; placing the cover over the screen; providing the pathway between the cover and the source of subatmospheric pressure for applying the subatmospheric pressure within the space defined by the cover; increasing the area of contact between the tissue and the screen by applying the subatmospheric pressure within the space defined by the cover; and releasing at least a portion of the at least one therapeutic or prophylactic agent to the increased area of contacted tissue.

Finally, many other features, objects and advantages of the present invention will be apparent to those of ordinary skill in the relevant arts, especially in light of the foregoing discussions, the following drawing and exemplary detailed description and the claims appended hereto.

### BRIEF DESCRIPTION OF THE DRAWINGS

Although the scope of the present invention is much broader than any particular embodiment, a detailed description of the preferred embodiment follows together with an illustrative figure, wherein like reference numerals refer to like components, and wherein:

FIG. 1 shows, in partially cut away perspective view, the preferred embodiment as applied to a mammalian wound site.

FIG. 2 is a flow chart of a process for uniformly coating a wound dressing with antimicrobial agents encapsulated in a polymer-based coating;

FIG. 3 is a schematic diagram of certain steps of the process of FIG. 2;

FIG. 4 is a schematic top plan view of a dressing coated using the process of FIG. 2 or FIG. 20 as applied to a wound site;

FIG. 4A is a schematic top plan view of an alternate embodiment of a dressing coated using the process of FIG. 2 or FIG. 20 as applied to a wound site of FIG. 4;

FIG. 5 is a side view of the dressing of FIG. 4 on a wound site in combination with a subatmospheric pressure therapeutic device;

FIG. 6 is a cross section of the dressing of FIG. 4 taken along line 6-6, illustrating the uniform coating of the dressing;

FIG. 7 is a schematic layout of one embodiment of the apparatus;

FIGS. 8A and 8B are pictorial representations of the housing of the pump and canister for the apparatus of FIG. 7;

FIGS. 9A and 9B are pictorial representations of the apparatus of FIG. 7 supported on a belt and harness respectively;

FIG. 10 is an exploded view of the housing showing the contents of the apparatus of FIG. 7;

FIGS. 11A to 11F show various views of a preferred form of the canister for the apparatus of FIG. 7 and a section of a multi-lumen tube;

FIGS. 12A to 12D show various views of a foam dressing connector for connecting the housing to the dressing;

FIG. 12E is a section of an alternative embodiment of the multi-lumen tube;

FIGS. 13A and 13B show a plan and perspective view of a surgical drape for use with the apparatus of FIG. 7 and FIG. 14;

FIG. 14 is a schematic layout of an alternative embodiment of the apparatus;

FIG. 15A is a perspective view of a fluid sampling port;

FIG. 15B is a perspective view of an alternative embodiment of a fluid sampling port;

FIG. 16A is a perspective view of the back portion of a pump housing for the apparatus of FIG. 14;

FIG. 16B is a perspective view of the front portion of a pump housing for the apparatus of FIG. 14;

FIGS. 17A and 17B are flow charts representing the preferred steps in the implementation of a power management system;

FIG. 18 is a flow chart illustrating the preferred steps in the implementation of pulse therapy;

FIG. 19 is a section view of an alternative embodiment of a cover for use with the apparatus of FIG. 7 and FIG. 14; and

FIG. 20 is a flow chart of a process for uniformly coating a foam or dressing with an antimicrobial metallic coating.

### DETAILED DESCRIPTION OF ILLUSTRATIVE EMBODIMENTS

Although those of ordinary skill in the art will readily recognize many alternative embodiments, especially in light of the illustrations provided herein, this detailed description is exemplary of the preferred embodiments of a subatmospheric pressure wound therapy system with provision for introduction of an agent, the scope of which is limited only by the claims appended hereto.

One embodiment provides a method for uniformly coating a wound dressing with polymers incorporating agents, such as Ag, utilizing a process and a wound dressing formed under the process. The method of uniform coating enables a user of the dressing to sever the predisposed dressing in any direction and still have all exposed surfaces uniformly coated with an antimicrobial agent sufficient to decontaminate the wound.

An alternative embodiment provides a method for uniformly coating a foam or dressing with a metal-based coating incorporating agents, such as Ag, and a dressing formed under the process. As with the polymer-based coating process, the metal-based coating process enables the user to sever the predisposed dressing in any direction and still have all exposed surfaces uniformly coated with the agent sufficient to treat the wound.

Silver serves herein as an exemplary antimicrobial agent, since the properties of silver allow it to be easily incorporated into both polymer-based coatings and into metal-based coatings. Other agents useful in alternative embodiments include, but are not limited to, therapeutic and prophylactic agents, such as antimicrobial agents, enzymatic debriders, anesthetic agents, chemotherapeutic agents, indicating agents, and growth factors. Antimicrobial agents include but are not limited to antibacterial agents such as antibiotic and bacteriostatic agents. Growth factors useful in embodiments discussed herein include, but are not limited to basic fibroblast growth factor, transforming growth factor, epidermal growth factor, platelet derived growth factor, insulin-like growth factor, keratinocyte growth factor, fibroblast growth factor, granulocyte macrophage colony stimulating factor, and granulocyte colony stimulating factor. A coating may incorporate single or multiple agents for release to the tissue and to the body-liquid drawn from the tissue. The coating contacts body-liquid and tissue, and releases the agent(s) in the presence of an aqueous environment.

A dressing or screen formed by the coating process is comprised of a substrate uniformly covered with the polymer-based or metal-based coating. The dressing or screen includes a plurality of flow ports or passages provided to allow gas and body-liquid to pass through for facilitating tissue healing. Surfaces of the plurality of ports or passages are also uniformly covered with the coating. The substrate may include, without limitation, material such as foam, yarn, film, filament, fiber, fabric, filler materials, or any combination thereof. The substrate material may be comprised of any substance capable of having the coating applied thereto, including without limitation, nylon, polyester, acrylic, rayon, cotton, polyurethane, other polymeric materials, cellulose materials, such as wood fiber, or any combination thereof. A foam portion of the dressing is preferably of open-celled, reticulated polyurethane, polyether, polyvinylacetate, or polyvinylalcohol construction, but other substitutions or modifications to the foam substrate are considered to be within the scope of this invention.

In one embodiment, a polyurethane foam is uniformly coated with a silver hydrogel polymer. The polymer coating itself contains PVP or Poly(vinyl-pyrrolidone), which is a water-soluble polymer with pyrrolidone side groups, typically used as a food additive, stabilizer, clarifying agent, tableting adjunct and dispersing agent. It is most commonly known as the polymer component of Betadine (a povidone-iodine formulation). In addition, the coating may contain Chitosan, which is a deacetylated derivative of chitin, a polysaccharide that is refined from shells of shrimps, crabs and other crustaceans. Chitosan has also been used in hemostatic dressings. The third optional component of the polymer is preferably Silver Sodium Aluminosilicate, which is silver salt powder with 20% active ionic silver by weight.

In a preferred embodiment, an apparatus and process for treating tissue is provided, wherein the foam or dressing formed by the polymer-based or metal-based coating processes discussed herein serves as the screen for use with a subatmospheric pressure tissue treatment device. The screen is placed in contact with the tissue and enclosed under a generally impermeable cover. The cover provides a substantially air-tight seal over the screen and the tissue, and defines a space over the tissue and under the cover. A liquid conduit is connected between a source of subatmospheric pressure and the cover to provide a pathway for applying a subatmospheric pressure within the space defined by the cover and for drawing interstitial and surface body-liquid therefrom.

When the subatmospheric pressure is applied to a tissue site, the screen compresses and conforms to the surface of the tissue as air is removed from within the space defined by the cover. Microdeformation of the tissue under the cover also occurs. These movements increase an area of contact between the screen and the tissue. In the aqueous environment within the space defined by the cover, the coating releases the agent, such as silver, directly to the increased area of contacted tissue. Increasing the area of contacted tissue brings the coating into direct contact with additional tissue, thereby maximizing the effectiveness of the agent release. In embodiments where the agent is silver, the coating releases silver ions directly to the contacted tissue to help reduce bacterial density on the area of contacted tissue.

As used herein, references to “grafting” are understood to generally refer to a process in which at least one therapeutic or prophylactic agent is bound to the surface of the carrier or substrate, including but not limited to covering the substrate with the coating comprising at least one therapeutic or prophylactic agent.

As used herein, references to “wound dressing,” “dressing,” and “foam” as a dressing, are understood to generally refer to the screen comprising the substrate uniformly covered with the coating. In a few instances, the terms have been used to refer to the substrate, or unmodified carrier material itself, but their meaning will obvious be to those skilled in the art. The screen is placed substantially over the tissue site to promote the growth of granulation tissue and also to prevent its overgrowth, and to release at least one therapeutic or prophylactic agent to the tissue site via the coating. As will be understood by those skilled in the art, the substrate may include, without limitation, material such as foam, yarn, film, filament, fiber, fabric, filler materials, or any combination thereof. The substrate may be comprised of any substance capable of having the coating applied thereto, including without limitation, nylon, polyester, acrylic, rayon, cotton, polyurethane, other polymeric materials, cellulose materials, such as wood fiber, or a combination thereof. Individual fibers are worked (woven, knitted, crocheted, felted, blown, etc.) into a fabric dressing. Foam dressing is preferably of open-celled, reticulated polyurethane, polyvinylalcohol, or polyvinylacetate construction, but other modifications to the foam dressing are considered to be within the scope of this invention.

As used herein, references to “drape” are understood to generally refer to a flexible sheet of construction that is generally body-liquid-impermeable. For purposes of this discussion, use of the term “impermeable” without further qualification, should be understood to generally refer to material and construction that is generally impermeable to body-liquid. Most particular examples include drapes such as those comprising an impermeable elastomeric material, such as a film, the underside of which is at least peripherally covered with a pressure-sensitive adhesive for providing a substantially air-tight seal with a second region of tissue surrounding the tissue site. Alternatively, drapes may be substituted with other covers while still appreciating certain aspects of the invention.

As used herein, references to “subatmospheric pressure” are understood to generally refer to a pressure less than the ambient atmospheric pressure outside the covered tissue site receiving treatment. In most cases, this subatmospheric pressure will be less than the atmospheric pressure at which the patient is located. Subatmospheric pressure tissue treatment may comprise a substantially continuous application of the subatmospheric pressure, where the subatmospheric pressure is relieved only to change the screen, or it can be practiced with the use of a cyclic application of the subatmospheric pressure in alternate periods of application and non-application, or it can be practiced by oscillating the pressure over time.

As used herein, references to “tissue” are understood to generally refer to an aggregation of similar cells or types of cells, together with any associated intercellular materials adapted to perform one or more specific functions including, but not limited to bone tissue, adipose tissue, muscle tissue, dermal tissue, vascular tissue, connective tissue, cartilage, tendons, and ligaments.

As used herein, references to “wound” and “wound site” are understood to generally refer to the tissue site, wherein the term “tissue site” is understood to generally refer to a region of tissue, including but not limited to a wound or defect located on or within any tissue. The term “tissue site” may further refer to the region of any tissue that is not necessarily wounded or defective, but is instead such as those in which it is desired to add or promote the growth of additional tissue. For example, the subatmospheric pressure tissue treatment may be used in certain tissue regions to grow additional tissue that may be harvested and transplanted to another tissue location.

As used herein, references to “wound fluids,” “wound exudates,” “fluid drainage,” or “fluids” or “liquid” related to the tissue site, are understood to generally refer to body-liquid, wherein the term “body-liquid” is understood to generally refer to any interstitial liquid in the tissues or liquid that has exuded from the tissue or its capillaries.

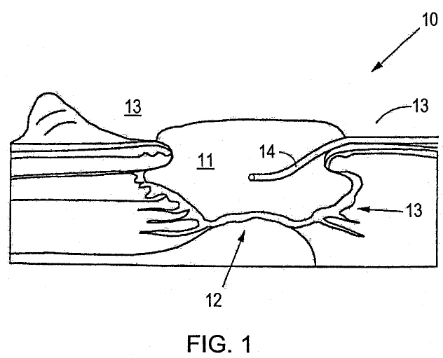

Referring now to FIG. 1, one embodiment 10 is shown to generally comprise a foam pad 11 for insertion substantially into a wound site 12 and a wound drape 13 for sealing enclosure of the foam pad 11 at the wound site 12. According to this embodiment, the foam pad 11 is placed in fluid communication with a source of subatmospheric pressure for promotion of wound healing. Additionally, the foam pad 11 is predisposed, through grafting or other techniques known to those of ordinary skill in the art, with basic fibroblast growth factor (bFGF), antimicrobials or other factors, also known to those of ordinary skill in the art, for the promotion of increased wound healing.

The foam pad 11, wound drape 13 and source of subatmospheric pressure are implemented as known in the prior art, each of which is detailed in U.S. patent application Ser. No. 08/951,832 filed Oct. 16, 1997. By this reference, the full disclosure of U.S. patent application Ser. No. 08/951,832 (“the ‘832 application”), including the claims and drawings, are incorporated herein as though now set forth in its entirety. Additionally, such a subatmospheric pressure wound therapy system is commercially available through KCI USA, Inc. of San Antonio, Tex., U.S.A. and/or its subsidiary companies through its V.A.C.® product line, as discussed below with reference to FIG. 5.

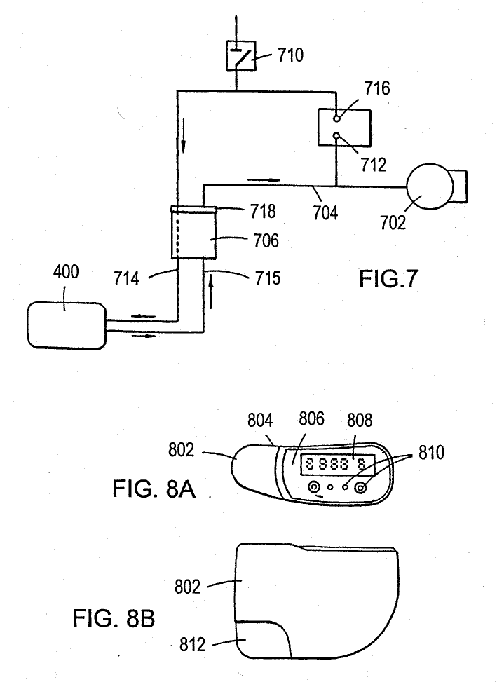

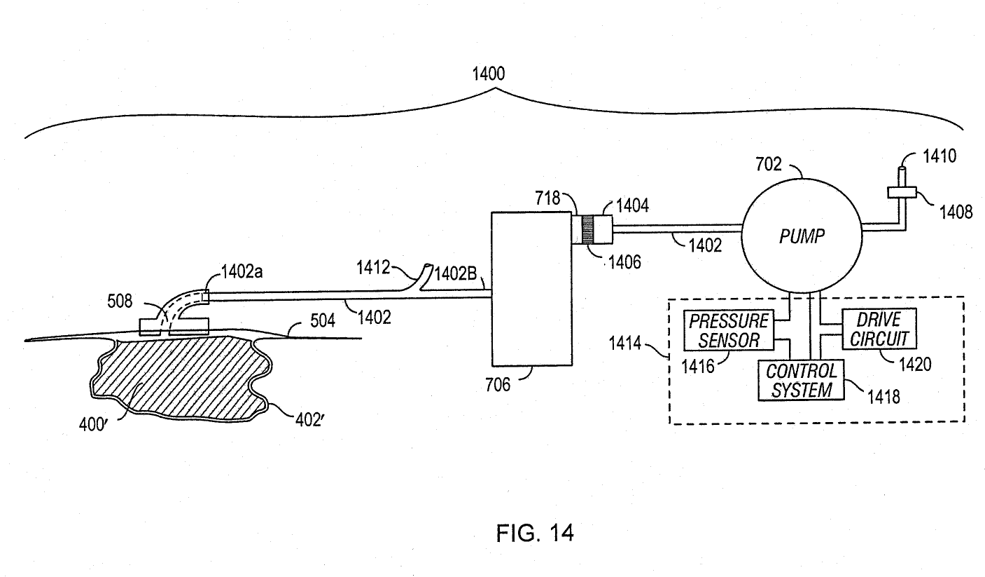

Alternative embodiments 10, generally comprising the foam pad 11, wound drape 13 and source of subatmospheric pressure as detailed in U.S. Pat. No. 6,142,982, issued on May 13, 1998 to Hunt, et al., and as detailed in U.S. Pat. No. 7,004,915 issued on Feb. 28, 2006 to Boynton, et al., are described and substantially set forth below with reference to FIGS. 7 to 13B and to FIGS. 14 to 18 respectively.

As detailed in the abovementioned patents and applications, the foam pad 11 preferably comprises a highly reticulated, open-cell polyurethane or polyether foam for good permeability of wound fluids while under subatmospheric pressure, but in this application may comprise a conventional sponge cellulose type dressing as necessary for introduction of the desired agent. As also detailed in the abovementioned patents and applications, the foam pad 11 is preferably placed in fluid communication, via a plastic or like material hose 14, with a source of subatmospheric pressure, which preferably comprises a canister safely placed under subatmospheric pressure through fluid communication, via an interposed hydrophobic filter, with a subatmospheric pressure pump. Finally, the abovementioned patents and applications also detail the wound drape 13, which preferably comprises an elastomeric material at least peripherally covered with a pressure sensitive, acrylic adhesive for sealing application over the wound site 12.

According to one embodiment, those components as are described in the abovementioned patents and applications are generally employed as known in the art with the exception that the foam pad 11 is modified as necessary for the introduction of a growth factor. Such growth factors as the basic fibroblast growth factor (bFGF) are known to accelerate wound healing due to their potent angiogenesis and granulation tissue formation activities. As has been demonstrated even with difficult to heal wounds, such as infected wounds, burn wounds and diabetic wounds, the resultant activities lead to the rapid reepithelialization and contraction of the wound. The combination of subatmospheric pressure therapy with growth factor introduction, through the modification of the foam pad 11 and predisposition thereof with the bFGF, is therefore thought to be an important contribution to the wound healing arts. Likewise, this method presents an excellent opportunity for the introduction to the wound site 12 of anti-microbial agents, whether alone or in combination with bFGF or other agents.

In the above embodiments discussed with reference to FIG. 1, the foam pad 11, modified and predisposed with therapeutic or prophylactic agents bound to the surface of the foam or dressing material, serves as the screen for use in delivering the at least one therapeutic or prophylactic agent to the tissue site in combination with the application of subatmospheric pressure for promotion of healing. Further, in the above embodiments, the wound site 12 serves as the tissue site, the wound drape 13 serves as the cover, the canister serves as a container, and the hose 14 serves as the liquid conduit.

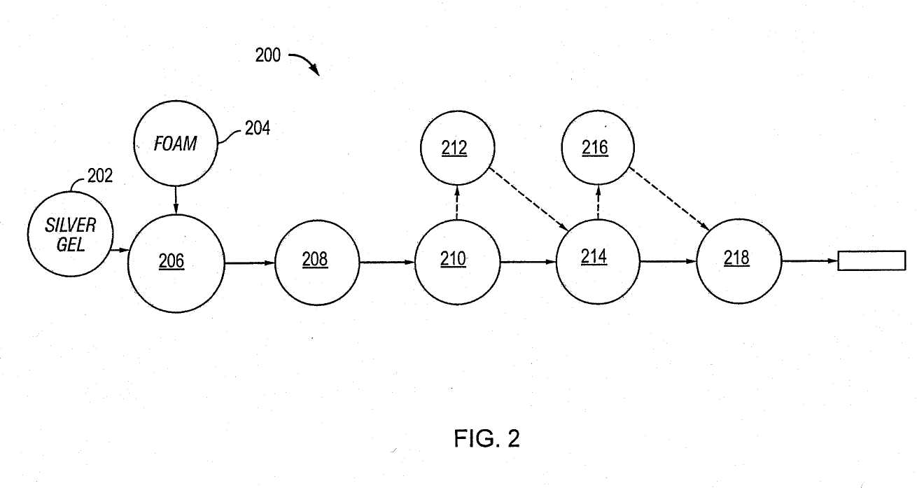

The at least one therapeutic or prophylactic agent may be bound to the foam or dressing substrate using the coating processes described in U.S. patent application Ser. No. 11/189,195 filed Jul. 26, 2005, and its copending continuation-in-part U.S. patent application entitled SYSTEM AND METHOD FOR USE OF AGENT IN COMBINATION WITH SUBATMOSPHERIC TISSUE TREATMENT filed Jul. 26, 2006, illustrated and substantially set forth below with reference to FIGS. 2 to 6 and FIG. 20, or other techniques known to those of ordinary skill in the art. By this reference, the full disclosure of U.S. patent application Ser. No. 11/189,195 (“the ‘195 application”), and its copending continuation-in-part U.S. patent application entitled SYSTEM AND METHOD FOR USE OF AGENT IN COMBINATION WITH SUBATMOSPHERIC TISSUE TREATMENT filed Jul. 26, 2006, including the claims and drawings, are incorporated herein as though now set forth in its entirety.

Referring next to FIG. 2, a method 200 for impregnating a foam with a silver polymer coating or antimicrobial coating is shown in the flow chart. First, a hydrophilic gel is combined with silver to create a coating solution, 202. The solution is then placed in a holding tank and continuously agitated in a closed, dark environment, 204. The dark environment is optional, but is included because of the light-sensitivity of silver. In a light-exposed environment, the foam may change color, which results in a non-aesthetic appearance. The foam, which may comprise reticulated polyurethane die-cut foam, is placed in the holding tank, 206. The foam is then saturated with the solution, which is accomplished through soaking or squeezing the foam, 208. Next, excess solution is removed from the foam, 210. Roller nips or similar devices may be utilized to control the amount of solution removed from the foam. Optionally, the weight of the saturated foam, while still wet, may be calculated, 212.

The foam is then placed in a convectional forced-air oven set to a predetermined temperature and time to completely dry the solution-coated foam, 214. Alternatively, to verify the dry condition of the foam, the weight of the foam may be checked again, 216. If light-sensitivity remains an issue, the foam can be packaged in a pouch with a low moisture vapor transmission rate (MVTR), which limits the exposure of the foam to light and to humidity, 218. The foam is now ready for use on such sites as partial thickness burns, traumatic wounds, surgical wounds, dehisced wounds, diabetic wounds, pressure ulcers, leg ulcers, flaps and grafts.

In one example, a foam made by the method described has achieved in-vitro efficacy on two common bacteria-staphylococcus aureus and pseudomonas aeruginosa, with a 20% silver salt load (4% silver by weight, though about 0.1% to about 6% has shown to be at least partially effective). The dressing maintains its effectiveness for 72 hours through a controlled and steady state release of ionic silver. Specifically, a diffusive gradient exists between the silver coating and the anionic rich outside environment that lead to disassociation and eventual transport of the silver ion. Using the above process, over a 6 log reduction or about 99.9999% of pathogenic bacteria have been eliminated between about 24 hours and about 72 hours.

The coating process can easily incorporate other additives, such as enzymatic debriders, anesthetic agents, growth factors and many other biopharmaceuticals. In addition, the coating can be formulated specific to coat thickness, although very thin coatings (about 2 to 10 micrometers) are preferable. The formulation can further be adapted to allow for large particle sizes and different release kinetics, such as concentration and rate and the duration of release.

The coating process can also easily incorporate other additives, singly or in combination. Those skilled in the art can easily adapt this process for polymer-coating other substrates previously listed, such as fiber or film, without undue experimentation.

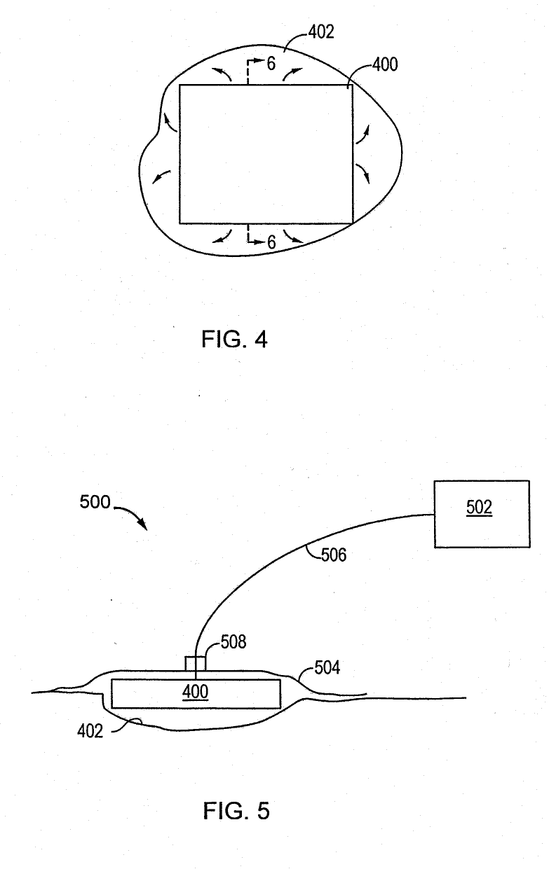

The uniform coating allows for delivery of silver ions both outside and within the foam. In this manner, not only is bacteria eliminated on the wound bed, but also within the dressing itself. As discussed below with reference to FIG. 4, this is particularly useful when using the dressing in combination with subatmospheric pressure therapy. Also, odor reduction is an added benefit of this method.

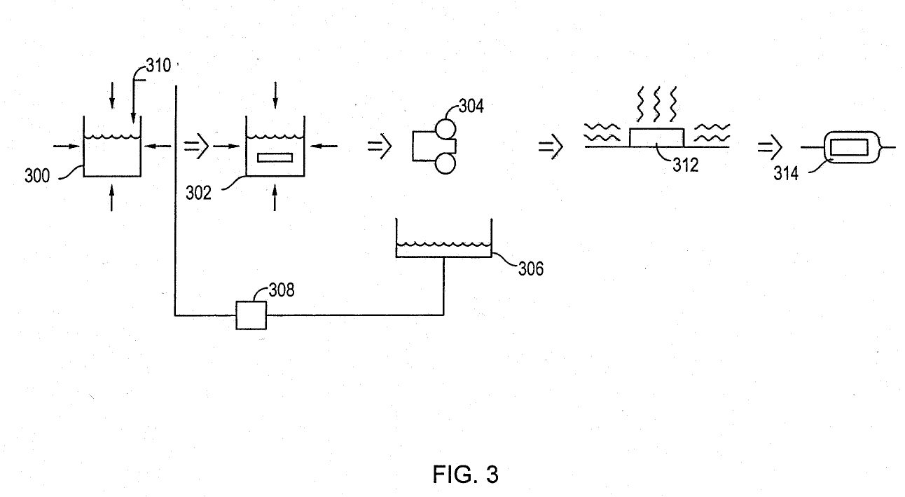

Referring now to FIG. 3, a schematic diagram of certain steps of the process 200 of FIG. 2 is shown. First, the solution of hydrophilic gel and the antimicrobial or other agent, such as silver, is shown in a tank subject to agitation, 300. Next, foam is inserted into the agitating tank, 302. After saturation, the foam is removed and fed through rollers or the like to remove excess solution, 304. The excess solution is captured, 306, and subjected to filtration by a filter sufficiently fine to rid particles from the solution and break apart any chunks of solution that may have formed during the process, 308. A 150-micron filter has been found to be effective during certain silver-solution coating experiments. The filtered solution is then returned to the tank for re-use, 310.

The foam from the removal step 304 is subjected to a convection oven for drying, 312. During certain silver-solution coating experiments, when the temperature of the oven is set at about 90° C., 20 minutes has been found to be an effective drying time. However, it is preferable to dry the foam for about at least 6 minutes to minimize any breakdown of coating. The foam is next packaged in appropriate containers, such as the MVTR pouch or similar containers for shipment to the user, 314.

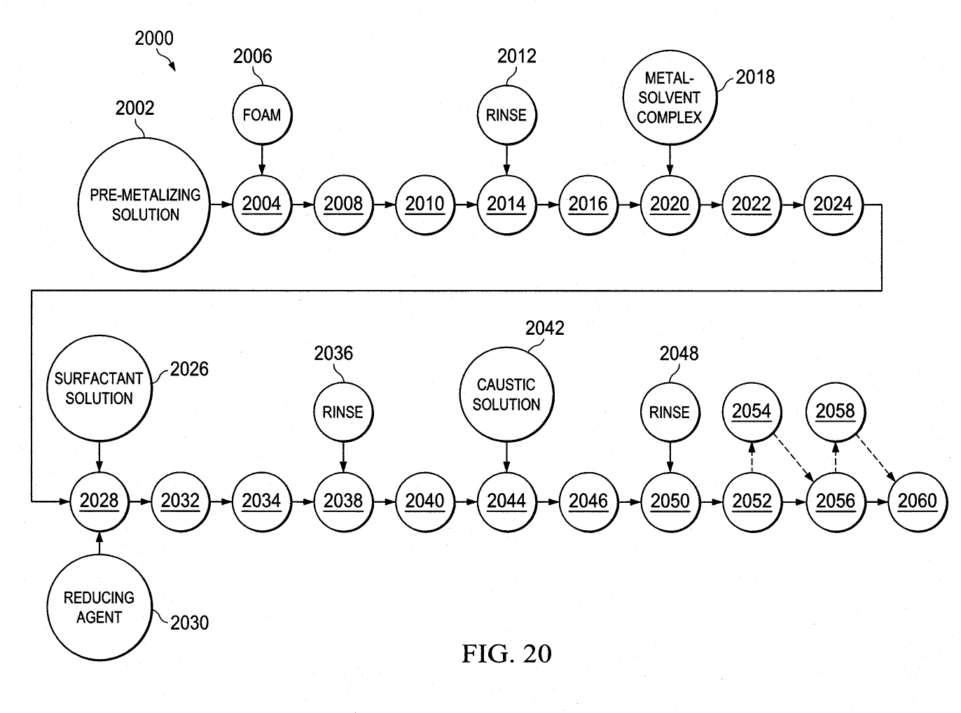

Referring now to FIG. 4, a schematic top plan view of a dressing 400 coated using the process of FIG. 2 or FIG. 20 as applied to a wound site 402 is shown. As indicated by the arrows, silver ions from the dressing 400 contact the wound site 402 and effectively eliminate bacteria formed thereon.

The uniform coating allows for delivery of silver ions both outside and within the dressing 400. Silver ions release from the uniform coating in the aqueous environment and diffuse to the tissue and into the body-liquid. Pathogens on the tissue, on the underside of the drape, and in the body-liquid that come into contact with the silver ions released from the coating on the outside of the dressing 400 are effectively eliminated. Reduction of bacterial density also occurs as application of the subatmospheric pressure through the dressing 400 effectively pulls body-liquid and accompanying pathogens through the uniformly coated dressing 400, bringing the pathogens into contact with the coating and silver ions within the dressing 400. Additionally, bacterial density within the container is reduced as body-liquid and accompanying silver ions are drawn into the container.

The embodiment of FIG. 4A includes dressing 400′ shown relative to the wound site 420′, and arrows representing silver ions migrating away from the dressing 400′ and contacting the wound site 402′, similar to the dressing 400, the wound site 402, and the arrows of FIG. 4. Whereas the dressing 400 of FIG. 4 has rectilinear edges, in FIG. 4A the edges of the dressing 400′ are adjusted to match the size and shape of the wound site 402′. As used herein, references to “dressing 400,” “pad 400,” “foam pad 400” and “foam pad 11” are understood to generally refer to the dressing 400′. Similarly, as used herein, references to “wound site 12” and “wound site 402” are understood to generally refer to the wound site 402′. In practice, the adjusting process is performed by a clinician at the wound site 402′ by severing the edges of a larger-sized dressing, in any direction necessary, to provide a smaller dressing 400′ shaped to match the overall shape of the wound site 402′.

When used in combination with subatmospheric pressure therapeutic devices, such as those commercialized by KCI USA, Inc. (and its affiliates) of San Antonio, Tex. as part of the V.A.C.® product line, the dressing 400 is particularly effective. FIG. 5 is a side view of the dressing 400 of FIG. 4 on a wound site 402 in combination with a subatmospheric pressure therapeutic device 500, which includes a control system 502, a drape 504 for covering the dressing 400 and wound site 402, a subatmospheric pressure hose 506 connected to the control system 502 and to the wound site 402 through the dressing 400, and a connector 508 for connecting the subatmospheric pressure hose 506 to the drape 504. Application of subatmospheric pressure by the control system 502 through the dressing 400 effectively pulls harmful pathogens through the uniformly coated dressing 400, thereby killing the pathogens. In addition, other surfaces of the dressing 400 in contact with the wound site 402 achieve the same result.

In the embodiment 500, the subatmospheric pressure therapeutic device 500 preferably serves as the “V.A.C. ATS®” or the “V.A.C. Freedom®” subatmospheric pressure tissue treatment device, commercially available from KCI USA, Inc. (and its affiliates) of San Antonio, Tex. The “V.A.C. ATS®” device is designed for higher acuity wounds and patients in acute care and long-term care facilities. The V.A.C. ATS® device is detailed in U.S. Pat. No. 7,004,915, issued to Boynton, et al., and set forth below with reference to FIGS. 14 to 18. The “V.A.C. Freedom®” device is a portable subatmospheric pressure tissue treatment device that allows patients to return to daily activities while continuing to receive subatmospheric pressure tissue treatment. The V.A.C. Freedoms® device is detailed in U.S. Pat. No. 6,142,982, issued to Hunt, et al., and set forth below with reference to FIGS. 7 to 13B. Suitable alternative subatmospheric pressure therapeutic devices may be the “V.A.C. Instill®” device, the “V.A.C.® Classic” device, the “Mini V.A.C.®” device, or any other “V.A.C.®” model device commercially available from KCI USA, Inc. (and its affiliates) of San Antonio, Tex. Additional suitable alternative devices, dressings and components may be those described in U.S. provisional patent application Ser. No. 60/765,548, entitled SYSTEMS AND METHODS FOR IMPROVED CONNECTION TO WOUND DRESSINGS IN CONJUNCTION WITH REDUCED PRESSURE WOUND TREATMENT SYSTEMS filed Feb. 6, 2006, the disclosure of which is incorporated by reference as though fully set forth herein. Such alternative V.A.C.® devices, dressings and components also may be generally represented by the subatmospheric pressure therapeutic device 500 and its dressings and components. Additionally, any of the abovementioned V.A.C.® devices, dressings and components also may be generally represented by the embodiment 10 of FIG. 1.

Further, in embodiment 500, the drape 504 serves as a cover, and is preferably the “V.A.C.® Drape” commercially available from KCI USA, Inc. (and its affiliates) of San Antonio, Tex. The subatmospheric pressure hose 506 serves as a liquid conduit, which combined with the connector 508 is preferably the “V.A.C. T.R.A.C.® Pad,” also commercially available from KCI USA, Inc. of San Antonio, Tex. Such components may also be represented by the wound drape 13 and the hose 14 of the embodiment 10 in FIG. 1.

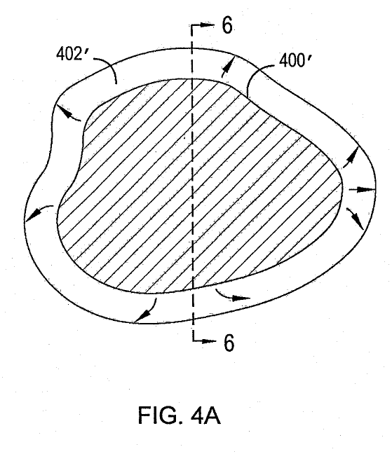

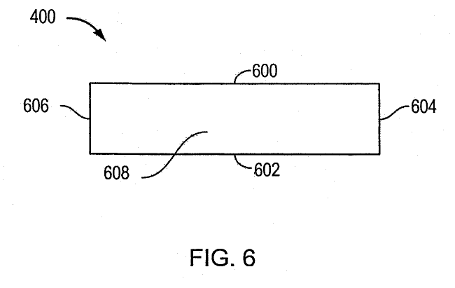

Referring now to FIG. 6, a cross-section of the dressing 400 of FIG. 4 taken along line 6-6 is shown, illustrating the uniform coating of the dressing 400. The dressing 400 has an upper surface 600, a lower surface 602, side surfaces 604, 606 and interior surface 608. All surfaces 600, 602, 604, 606, and 608, are coated with the silver coating, thereby providing an effective barrier to any pathogens that directly contact the surfaces or are indirectly exposed thereto by silver ions migrating away from the dressing 400.

One embodiment of the subatmospheric pressure therapeutic device 500 of FIG. 5 is described in U.S. Pat. No. 6,142,982, issued to Hunt, et al. on May 13, 1998, illustrated and substantially set forth below in FIGS. 7, 8A and 8B, 9A and 9B, 10, 11A to 11F, 12A to 12E, and 13A and 13B, and incorporated herein as though fully set forth. A preferred apparatus and process for detecting variations in application of the subatmospheric pressure within the space defined by the cover and for applying intermittent subatmospheric pressure therein is described below with reference to Hunt et al., and clarified with Boynton et al., further below.

Referring to the drawings, the portable therapeutic apparatus comprises a housing 802 (best shown in FIGS. 8A and 8B), having rounded corners and a side 804 which is concavely curved in order to fit comfortably to the wearer's body. The shaping of the housing with curved surfaces is to avoid sharp corners or edges that could dig in to the user or his caregiver. The upper surface 806 is generally flat and has an LCD screen 808 on which details such as applied pressure can be displayed. Control buttons 810 are provided to adjust pressures and treatment intervals. Provision is made for housing a canister within the housing and a snap release cover 812 is arranged for removing or introducing the canister.

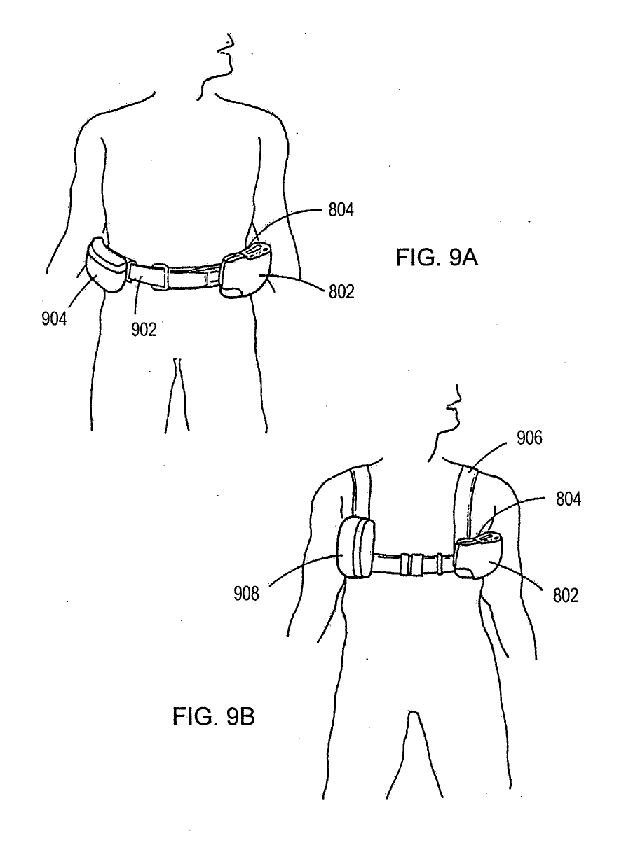

FIGS. 9A and 9B show schematically ways in which the housing 802 may be supported on the patient's body. In FIG. 9A the housing 802 is supported on a belt 902 and its weight is balanced by a similarly rounded casing 904 containing a rechargeable battery pack. FIG. 9B shows an alternative arrangement in which the housing is supported on a harness 906 and again a battery pack is contained in a housing 908, also supported on the harness.

FIG. 10 shows an exploded view of the housing 802 indicating the main components within the housing. The housing consists of front and rear shell moldings 1001 and 1002 having an external belt clip 1004 for attachment to a belt or harness.

Within housing shell 1001 is located a subatmospheric pressure pump 702 with associated electric motor 702A and the pump is connected by a silicon rubber tube 704 to a canister spigot 1006A in a compartment 1008 for the canister 706. Also connected to a second canister spigot 1006B via a tube 708 is a pressure relief valve 710 and both tubes 704 and 708 are connected via T-connectors T to pressure transducers (not shown). A microprocessor 1010 is mounted on a PCB board 1012 and a membrane assembly 1014 incorporates an LCD indicator and control buttons.

The apparatus may include means for recording pressures and treatment conditions given to a particular patient which may be printed out subsequently by the physician. Alternatively, the equipment may include a modem and a telephone jack so that the conditions under which the patient has been treated can be interrogated by the physician from a distant station.

Canister 706 is a push fit into the cavity 1008 and its lower end is supported in a cover 1016. The cover 1016 incorporates fingers 1018 which are releasably engageable with lips 1020 to hold the canister in position. The canister and the latch mechanism is arranged so that when the latch is engaged, the spigots 1006A and 1006B are in sealing engagement or abutment with tubular protrusions 1022 and 1024 formed in the top of the canister.

The method of operation of the apparatus can be appreciated from the schematic layout in FIG. 7, in which the canister 706 is connected via tube 715 to a porous dressing 400 at the wound site. Subatmospheric pressure is applied to the wound site via the canister by a tube 704, connected to the pump 702. The pressure in the tube 704 is detected by the transducer 712.

A second tube 714 is connected to the wound site 402 at one end, and also to a pressure relief valve 710 and to a second transducer 716. Tubes 714 and 715 can be combined in a multi-partitioned tube in manner to be described later. By means of tube 714 and transducer 716 the pressure at the wound site can be measured or monitored. A filter 718 is placed at or close to the outlet end of the canister 706 to prevent liquid or solid particles from entering the tube 704. The filter is a bacterial filter which is hydrophobic and preferably also lipophobic. Thus, aqueous and oily liquids will bead on the surface of the filter. During normal use there is sufficient air flow through the filter such that the pressure drop across the filter is not substantial.

As soon as the liquid in the canister reaches a level where the filter is occluded, a much increased subatmospheric pressure occurs in tube 704 and this is detected by transducer 712. Transducer 712 is connected to circuitry which interprets such a pressure change as a filled canister and signals this by means of a message on the LCD and/or buzzer that the canister requires replacement. It may also automatically shut off the working of the pump.

In the event that it is desired to apply intermittent subatmospheric pressure to the wound site, a pressure relief valve 710 enables the pressure at the wound site to be brought to atmospheric pressure rapidly. Thus, if the apparatus is programmed, for example, to relieve pressure at 10 minute intervals, at these intervals valve 710 will open for a specified period, allow the pressure to equalize at the wound site and then close to restore the subatmospheric pressure. It will be appreciated that when constant subatmospheric pressure is being applied to the wound site, valve 710 remains closed and there is no leakage from atmosphere. In this state, it is possible to maintain subatmospheric pressure at the wound site without running the pump continuously, but only from time to time, to maintain a desired level of subatmospheric pressure (i.e. a desired pressure below atmospheric), which is detected by the transducer 712. This saves power and enables the appliance to operate for long periods on its battery power supply.

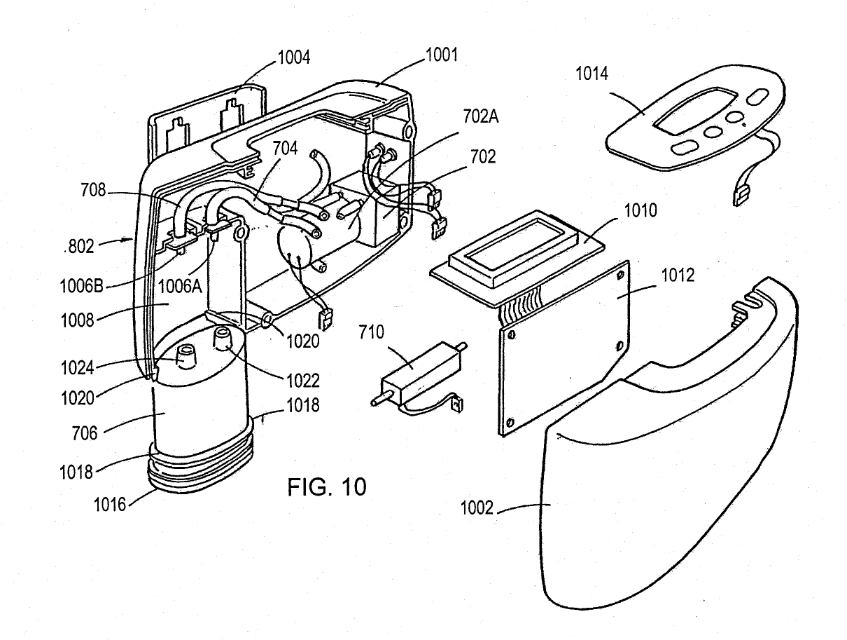

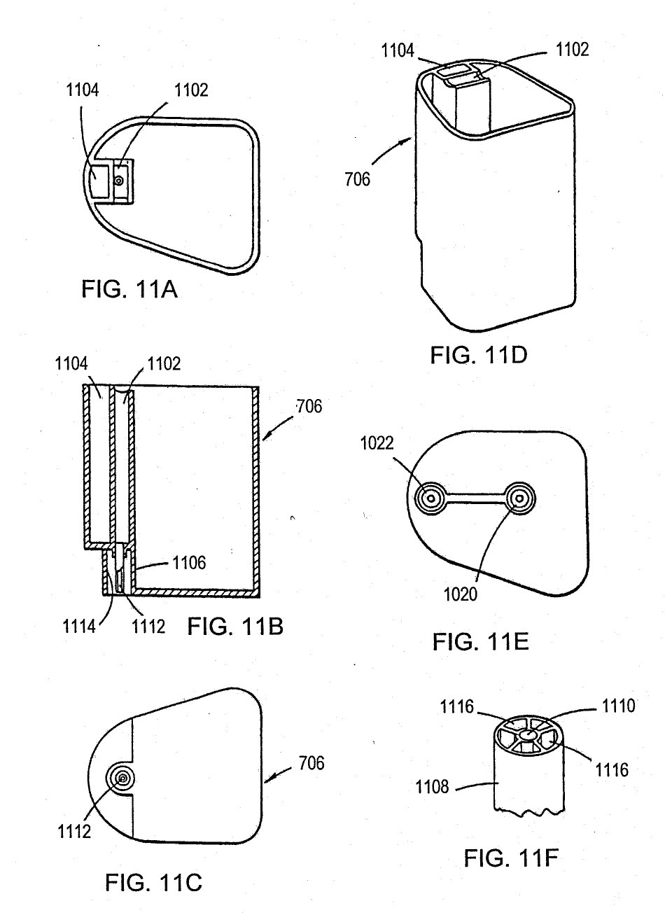

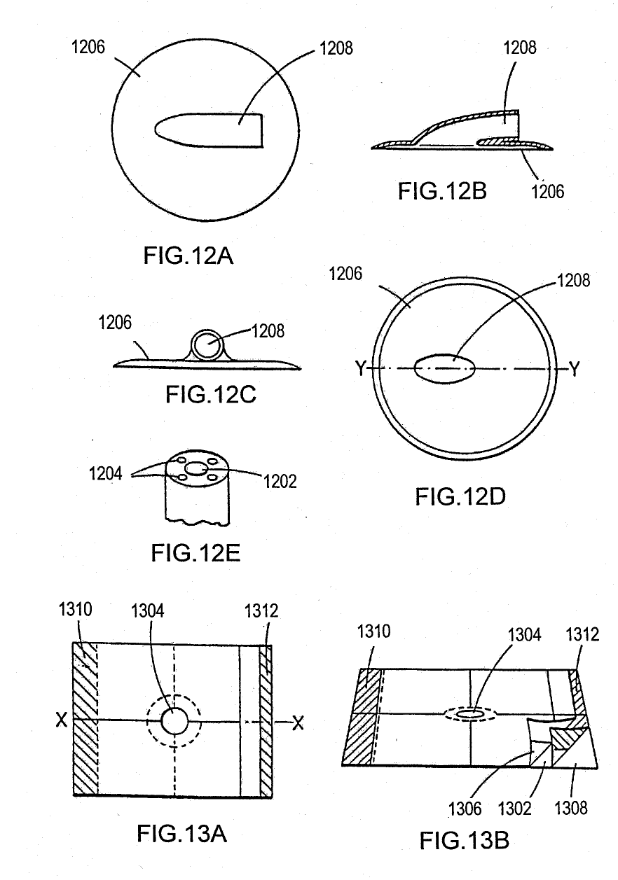

Instead of running two separate tubes to the wound site, it is preferable to contain tubes 714 and 715 in a single tube which is connected through the canister. Thus, for example, tubes 704 and 715 may comprise an internal tube surrounded by an annular space represented by tube 714. This is illustrated in FIGS. 11A to 11F and in a modified form in FIG. 12E.

In an alternative embodiment, the multi-lumen tube may be constructed as shown in FIG. 12E. In this embodiment, the internal bore 1202 comprises the line 715 (see FIG. 7) and is used to extract fluids from the wound site. Air flow (represented by line 714 in FIG. 7) passes down conduits 1204 located within the walls of the tube. By spacing the conduits 1204 at 90 degree intervals around the tube, the risk of arresting the air flow by kinking or twisting the multi-lumen tube is minimized.

FIG. 11E is a plan view of the top of a preferred shape of the canister, the generally triangular shape in section being chosen to fit better the space within cavity 1008 (see FIG. 10). Tubular protrusions on the top of the canister are connected internally of the canister with respectively conduits 1102 and 1104 (see sectional view of FIG. 11B), thus maintaining a separation between the tubes which are represented by lines 704 and 714 in FIG. 7. At the base of the canister, a molding 1106 facilitates connection to a multi-partitioned tube 1108 shown in FIG. 11F. Tube 1108 has a central bore 1110 that is sized to fit over a spigot 1112 in molding 1106. At the same time, the external wall of tube 1108 seals against the inner wall 1114 of molding 1106. Thus, compartment 1102 will connect with central bore 1110 and the compartment 1104 will connect with the annular spaces 1116 of tube 1108. In this way, a conduit 1116 corresponds with line 714 and central bore 1110 with line 715 as shown in FIG. 7.

The partitioned tube need not continue all the way to the wound site 402, but can be connected to a short section of single bore tube close to the wound site.

In the event of an air leak in the dressing at the wound site 402, this can be detected by both transducers 712 and 716 reading insufficient subatmospheric pressure for a specific time period, and then triggering a leak alarm, i.e. a message on the LCD, preferably also with an audible warning.

Typically, the pump 702 is a diaphragm pump but other types of pump and equivalent components to those specifically employed may be substituted.

FIGS. 12A-12D show various views of a connector for attaching the multi-lumen tube at the wound site. FIGS. 13A and 13B show a plan and perspective view of a surgical drape for attaching the connector to a porous dressing at the wound site. The connector comprises a molded plastics disc-like cup 1206 having a centrally positioned spout 1208. The spout 1208 is sized to accept, as a closely sliding fit, the end of a multi-lumen tube, e.g. of the kind shown in FIG. 11F or 12E. In use, a porous dressing is cut to correspond with the extent of the wound and pressed onto the wound as shown in FIG. 10 of our PCT application WO 96/05873. Instead of introducing the lumen into the foam dressing, the cup 1206 is pressed onto the porous dressing and secured by a surgical drape. However, if desired, the end of the lumen can be passed into the spout and additionally pressed into the foam. A surgical drape, such as shown in FIGS. 13A and 13B, can be used to secure the connector, lumen and dressing. The drape comprises a polyurethane film 1302 coated on one side with a pressure-sensitive acrylic resin adhesive. A hole 1304 is cut through all layers of the drape and the hole is dimensioned to correspond approximately with the outer cross-section of the spout 1208. Film 1302 has an overall size that allows it to be adhered to the patient's skin around the wound site while, at the same time, securing the connector to the porous dressing. A sufficient overlap around the wound is provided so that an airtight cavity is formed around the wound.

In an alternative form, the drape can be made in two parts, e.g. by cutting along the line X-X in FIG. 13A. With this arrangement, the wound can be sealed by overlapping two pieces of surgical drape so that they overlap each other along a line Y-Y as shown in FIG. 12D.

The surgical drape may include a protective film 1306, e.g. of polyethylene, and a liner 1308 that is stripped off prior to use to expose the pressure-sensitive adhesive layer. The polyurethane film may also include handling bars 1310, 1312, which are not coated with adhesive, to facilitate stretching of the film over the wound site. The dressing is preferably a pad of porous, flexible plastics foam, e.g. reticulated, open intercommunicating cellular flexible polyurethane foam, especially of the kind described in the above-mentioned PCT application WO 96/05873.

Alternatively, a reticulated intercommunicating cellular foam made from flexible polyvinylacetate or polyvinylalcohol foam may be used. The latter is advantageous because it is hydrophilic. Other hydrophilic open celled foams may be used.

In another method of therapy, the foam dressing may be sutured into a wound after surgery and the foam dressing connected to the pump unit by the multi-lumen catheter. Subatmospheric pressure can then be applied continuously or intermittently for a period determined by the surgeon, e.g. from about 6 hours to 4 to 5 days. After this period, the dressing is removed and the wound re-sutured. This therapy improves the rate of granulation and healing of wounds after surgery.

In the foregoing embodiments described with reference to Hunt, et al., the LCD screen 808, microprocessor 1010, and PCB board 1012 combine to serve as a controller; the subatmospheric pressure pump 702 serves as the source of subatmospheric pressure; the tubes 704 and 715 together serve as the liquid conduit; the transducer 712 serves as the pump pressure transducer; the tubes 708 and 714 together serve as the pressure detection conduit, and the transducer 716 serves as the tissue pressure transducer.

As described above, the tubes 714 and 715 may be contained in one tube to serve as the multi-lumen conduit, wherein the internal bore 1202 serves as a liquid lumen and conduits 1204 serve as pressure detection lumen. Further, the canister 706 serves at the container; the surgical drape serves as the cover; the dressing 400 serves as the screen; and the wound site 402 serves as the tissue site. After the screen is placed in contact with the tissue site, the cover is positioned to enclose the screen, defining the space under the cover and over the tissue site for application of the subatmospheric pressure. It is contemplated that the device may also include wireless communication equipment to allow physicians to remotely access records of the conditions under which the patient has been treated.

An alternative embodiment of the subatmospheric pressure therapeutic device 500 of FIG. 5 is described in U.S. Pat. No. 7,004,915, issued to Boynton, et al., on Feb. 28, 2006, illustrated and substantially set forth below in FIGS. 14, 15A and 15B, 16A and 16B, 17A and 17B, and 18, whose reference is incorporated herein as though fully set forth.

A preferred apparatus and process for detecting whether a container is filled with the body-liquid drawn from within the space defined by the cover, and for preventing the body-liquid from contaminating the source of subatmospheric pressure is set forth below with reference to Boynton et al. A preferred apparatus and process for oscillating application of the subatmospheric pressure over time is also described below with reference to Boynton et al.

The following embodiment is a vacuum assisted system for stimulating the healing of tissue.

Referring now to FIG. 14 in particular, there is illustrated the primary components of a system that operates in accordance with an alternative embodiment. This embodiment 1400 includes a foam pad 400′ for insertion substantially into a wound site 402′ and a wound drape 504 for sealing enclosure of the foam pad 400′ at the wound site 402′. The foam pad 400′ may be comprised of a polyvinyl alcohol (PVA) open cell polymer material, or other similar material having a pore size sufficient to facilitate wound healing. A pore density of greater than 38 pores per linear inch is preferable. A pore density of between 40 pores per linear inch and 50 pores per linear inch is more preferable. A pore density of 45 pores per linear inch is most preferable. Such a pore density translates to a pore size of approximately 400 microns.

Addition of an indicating agent, such as crystal violet, methylene blue, or similar agents known in the art causes a color change in the foam 400′ when in the presence of a bacterial agent. As such, a user or health care provider can easily and readily ascertain if an infection is present at the wound site 402′. It is contemplated that the indicating agent may also be placed in line of the conduit 1402, between the wound site 402′ and the canister 706. In such a configuration (not shown), the presence of bacterial contaminants in the wound site 402′, could be easily and readily ascertained without disturbing the wound bed, as there would be a nearly immediate color change as bacterially infected wound exudates are drawn from the wound site 402′ and through the conduit 1402 during application of subatmospheric pressure.

It is also contemplated that the foam pad 400′ may be coated with a bacteriostatic agent. Addition of such an agent, would serve to limit or reduce the bacterial density present at the wound site 402′. The agent may be coated or bonded to the foam pad 400′ prior to insertion in the wound site, such as during a sterile packaging process. Alternatively, the agent may be injected into the foam pad 400′ after insertion in the wound site 402′.

After insertion into the wound site 402′ and sealing with the wound drape 504, the foam pad 400′ is placed in fluid communication with a subatmospheric pressure source 702 for promotion of wound healing and secondarily, fluid drainage, as known to those of ordinary skill in the art. The subatmospheric pressure source 702 may be a portable electrically powered pump, or other suitable subatmospheric pressure source.

According to one embodiment, the foam pad 400′, wound drape 504, and subatmospheric pressure source 702 are implemented as known in the prior art, with the exception of those modifications detailed further herein.

The foam pad 400′ preferably comprises a highly reticulated, open-cell polyurethane or polyether foam for effective permeability of wound fluids while under subatmospheric pressure. The pad 400′ is preferably placed in fluid communication, via a plastic or like material conduit 1402, with a canister 706 and a subatmospheric pressure source 702. A first hydrophobic membrane filter 718 is interposed between the canister 706 and the subatmospheric pressure source 702, in order to prevent wound exudates from contaminating the subatmospheric pressure source 702. The first filter 718 may also serve as a fill-sensor for canister 706. As fluid contacts the first filter 718, a signal is sent to the subatmospheric pressure source 702, causing it to shut down. The wound drape 504 preferably comprises an elastomeric material at least peripherally covered with a pressure sensitive adhesive for sealing application over the wound site 402′, such that a subatmospheric pressure seal is maintained over the wound site 402′. The conduit 1402 may be placed in fluidic communication with the foam 400′ by means of an appendage 508 that can be adhered to the drape 504.

According to a preferred embodiment, a second hydrophobic filter 1404 is interposed between the first filter 718 and the subatmospheric pressure source 702. The addition of the second filter 1404 is advantageous when the first filter 718 is also used as a fill sensor for the canister 706. In such a situation, the first filter 718 may act as a fill sensor, while the second filter 1404 further inhibits contamination of wound exudates into the subatmospheric pressure source 702. This separation of functions into a safety device and a control (or limiting) device, allows for each device to be independently engineered. An odor vapor filter 1406, which may be a charcoal filter, may be interposed between the first filter 718 and the second filter 1404, in order to counteract the production of malodorous vapors present in the wound exudates. In an alternate embodiment (not shown), the odor vapor filter 1406 may be interposed between the second hydrophobic filter 1406 and the subatmospheric pressure source 702. A second odor filter 1408 may be interposed between the subatmospheric pressure source 702 and an external exhaust port 1410, in order to further reduce the escape of malodorous vapors. A further embodiment allows for first 718 and second filters 1404 to be incorporated as an integral part of the canister 706 to ensure that the filters 718, 1404, at least one of which are likely to become contaminated during normal use, are automatically disposed of in order to reduce the exposure of the system to any contaminants that may be trapped by the filters 718 and 1404.

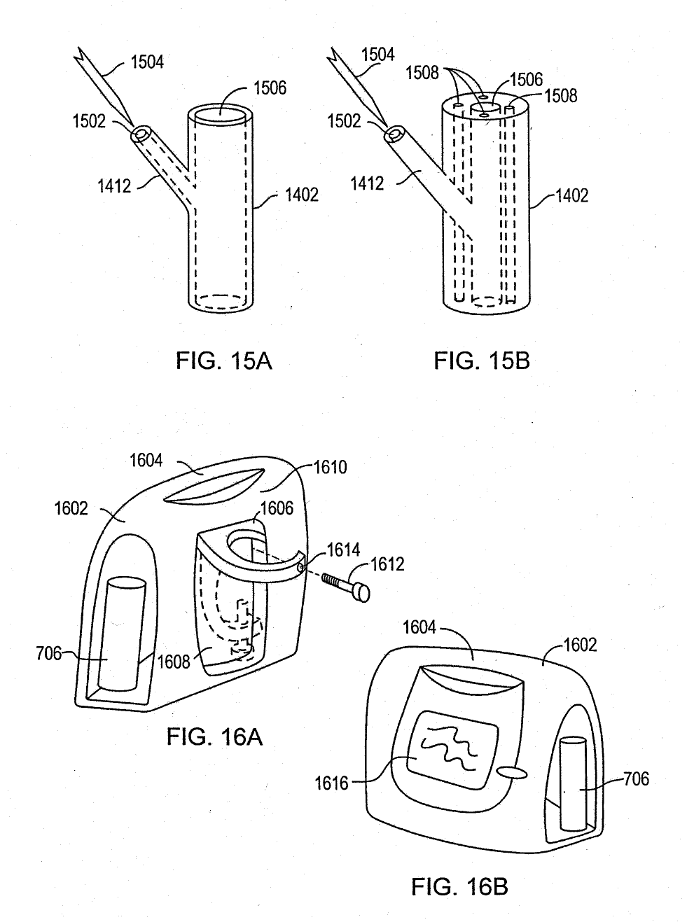

A means for sampling fluids may also be utilized by providing a resealable access port 1412 from the conduit 1402. The port 1412 is positioned between the distal end 1402a of the conduit 1402 and the proximal end 1402b of the conduit 1402. The port 1412, as further detailed in FIGS. 15A and 15B, is utilized to allow for sampling of fluids being drawn from the wound site 402′ by the application of subatmospheric pressure. Although the port 1412 is shown as an appendage protruding from the conduit 1402, it is to be understood that a flush mounted port (not shown) will serve an equivalent purpose. The port 1412 includes a resealable membrane 1502 that after being punctured, such as by a hypodermic needle, the seal is maintained. Various rubber-like materials known in the art for maintaining a seal after puncture can be utilized.

The process by which wound fluids are sampled comprises penetrating the membrane 1502 with a fluid sampler 1504, such as a hypodermic needle or syringe. The sampler 1504 is inserted through the membrane 1502 and into the port 1412 until it is in contact with wound fluids flowing through the inner lumen 1506 of the conduit 1402. As illustrated in FIG. 15B, and further described in U.S. Pat. No. 6,142,982, issued to Hunt, et al. on May 13, 1998, and whose reference is incorporated herein as though fully set forth, the inner lumen 1506 may be surrounded by one or more outer lumens 1508. The outer lumens 1508 may serve as pressure detection conduits for sensing variations in pressure at the wound site 402′. In an alternative embodiment (not shown), the outer lumen or lumens 1508 may act as the subatmospheric pressure conduit, while the inner lumen 1506 may act as the pressure detection conduit. In this embodiment, the fluid sampling port 1412, communicates only with the inner lumen 1506, so as not to interfere with pressure detection that may be conducted by the outer lumens 1508. In an alternate embodiment (not shown) in which the outer lumen 1508 serves as the subatmospheric pressure conduit, the fluid sampling port 1412 communicates with the outer lumen 1508.

The subatmospheric pressure source 702 may consist of a portable pump housed within a housing 1602, as illustrated in FIGS. 16A and 16B. A handle 1604 may be formed or attached to the housing 1602 to allow a user to easily grasp and move the housing 1602.

According to one embodiment, a means for securing the housing 1602 to a stationary object, such as an intravenous fluid support pole for example, is provided in the form of a clamp 1606. The clamp 1606, which may be a G-clamp as known in the art, is retractable, such that when not in use is in a stored position within a recess 1608 of the housing 1602. A hinging mechanism 1610 is provided to allow the clamp 1606 to extend outward from the housing 1602, to up to a 90 degree angle from its stored position. An alternative embodiment (not shown) allows the clamp 1606 to be positioned at up to a 180 degree angle from its stored position. The hinging mechanism 1610 is such that when the clamp 1606 is fully extended, it is locked in position, such that the housing 1602 is suspended by the clamp 1606. A securing device 1612, such as a threaded bolt, penetrates through an aperture 1614 of the clamp 1606, to allow the clamp 1606 to be adjustably secured to various stationary objects of varying thickness.

Alternatively, the securing device 1612, may be comprised of a spring actuated bolt or pin, that is capable of automatically adjusting to various objects, such as intravenous fluid support poles, having varying cross-sectional thicknesses.

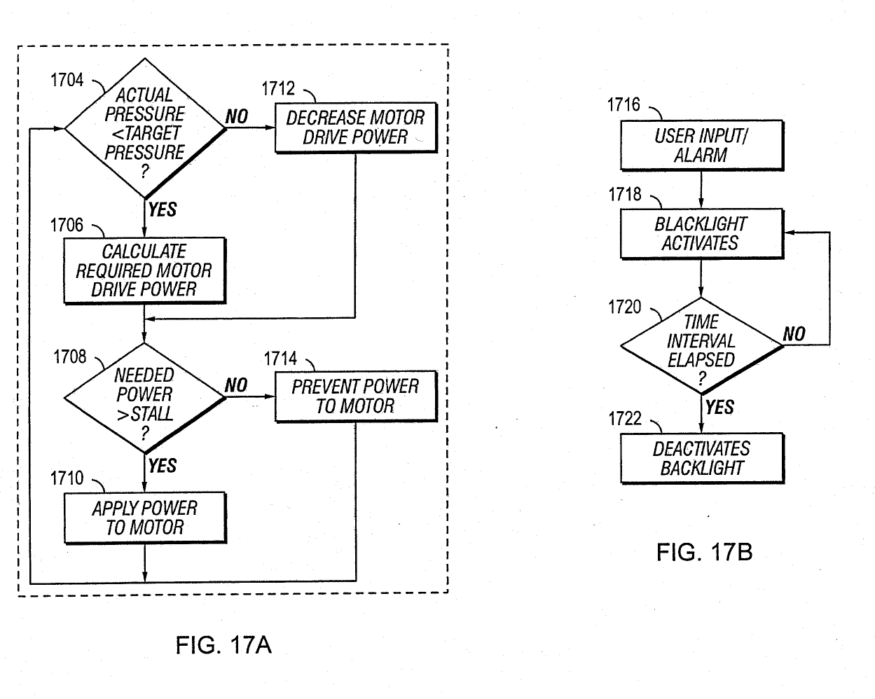

One embodiment also allows for management of a power supply to the subatmospheric pressure source 702, in order to maximize battery life when a direct current is utilized as a power supply. In a preferred embodiment, as illustrated in the flow chart of FIG. 17A, a motor control 1702 determines if the actual pressure is less than or equal to a target pressure 1704. If the actual pressure is less than the target pressure, a tentative motor drive power required to reach the target pressure is calculated 1706. If the tentative motor drive power required to reach the target pressure is greater or equal to the stall power 1708, the tentative motor drive power is actually applied to the motor 1710. If the actual pressure is greater than the target pressure, the tentative motor drive power is decreased and a determination is made as to whether additional power is needed to overcome the stall power 1712. If it is determined that the tentative power is inadequate to overcome the stall power, the tentative power is not supplied to the motor 1714. If the tentative power is adequate to overcome the stall power, the tentative power is actually applied to the motor 1710. The motor control 1702 functions as a closed loop system, such that the actual pressure is continuously measured against the predetermined target pressure. The advantage of such a system is that it prevents power from being supplied to the motor when it is not necessary to maintain the target pressure specified for V.A.C.® therapy. Accordingly, battery life is extended because power is not needlessly used to power the motor when it is not necessary.

Battery life is further extended, as illustrated in the flow chart shown in FIG. 17B, by providing a means, such as an integrated software program in a computer processor, for automatically disengaging a backlight of the visual display 1616 of the embodiment 1400 (as seen in FIG. 16B). User input of information 1716, such as target pressure desired, or duration of therapy, activates 1718 a backlight of the visual display 1616 shown in FIG. 16B. User input 1716 may also be simply touching the visual display 1616, which may be a touch activated or a pressure sensitive screen as known in the art. Activation of an alarm 1716 may also activate 1718 the backlight of the display 1616. An alarm may be automatically activated if an air leak is detected at the wound site 402′. Such a leak may be indicated by a drop or reduction in pressure being detected at the wound site 402′. The backlight remains active until a determination is made as to whether a preset time interval has elapsed 1720. If the time interval has not elapsed, the backlight remains active 1718. If the time interval has elapsed, the backlight is automatically extinguished 1722, until such time as the user inputs additional information, or an alarm is sounded 1716.

Referring now back to FIG. 14, battery life is further extended by means of a variable frequency pump drive system 1414, when the pump 702 is an oscillating pump. The pump drive system 1414 consists of a pressure sensor 1416, a control system 1418, and a variable frequency drive circuit 1420. In one embodiment the pressure sensor 1416 measures the pressure across the pump, which is relayed to the control system 1418. The control system 1418 determines the optimum drive frequency for the pump 702 given the pressure measured and relayed by the pressure sensor 1416. The optimum drive frequency for the pump 702 may be determined by the control system 1418 either repeatedly or continuously. The control system 1418 adjusts the variable frequency drive circuit 1420 to drive the pump at the optimum frequency determined by the control system 1418.

The use of the variable frequency pump drive system 1414 allows the pressure of the pump 702 to be maximized. In tests on sample oscillating pumps, the maximum pressure achieved was doubled by varying the drive frequency by only 30%. Additionally, the system 1414 maximizes flow rate over the extended frequency range. As a result, performance of the pump 702 is significantly improved over existing fixed frequency drive system pumps without increasing the pump size or weight. Consequently, battery life is further extended, thus giving the user greater mobility by not having to be tethered to a stationary power source. Alternatively, a similar performance level to the prior art fixed frequency drive system pumps can be achieved with a smaller pump. As a result, patient mobility is improved by improving the portability of the unit.

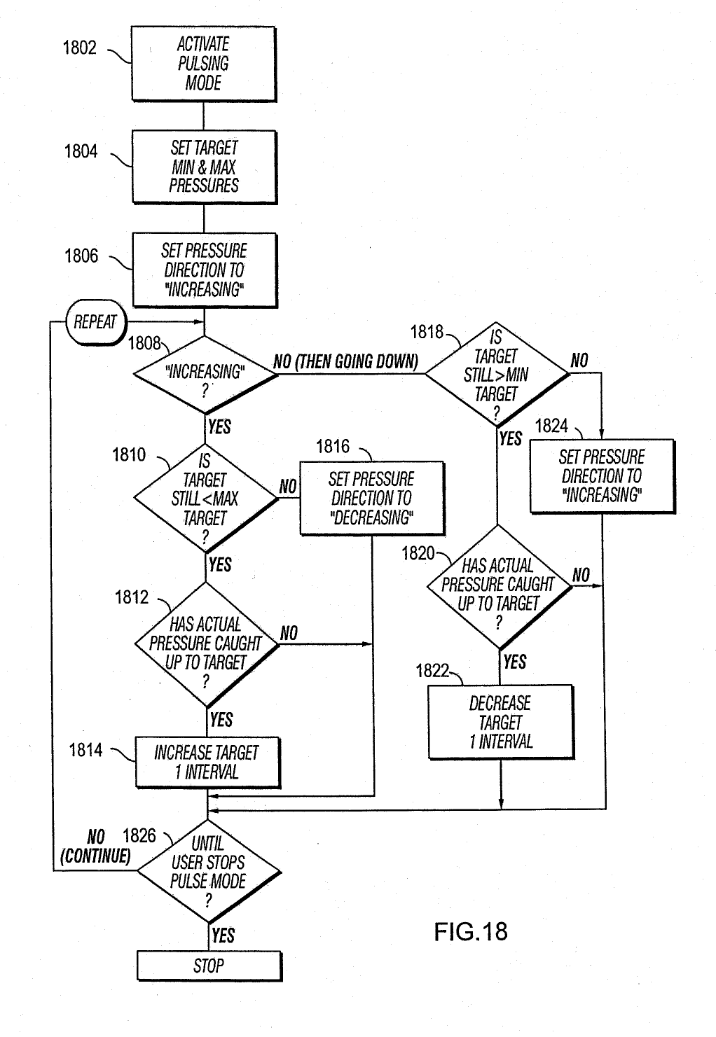

Another embodiment increases the stimulation of cellular growth by oscillating the pressure over time, as illustrated in the flow chart of FIG. 18. Such an oscillation of pressure is accomplished through a series of algorithms of a software program, utilized in conjunction with a computer processing unit for controlling the function of the subatmospheric pressure source or pump. The program is initialized when a user, such as a health care provider, activates the pulsing mode of the pump 1802. The user then sets a target pressure maximum peak value and a target pressure minimum peak value 1804. The software then initializes the pressure direction to “increasing” 1806. The software then enters a software control loop. In this control loop, the software first determines if the pressure is increasing 1808.

If the actual pressure is increasing in test 1808, a determination is then made as to whether a variable target pressure is still less than the maximum target pressure 1810. If the variable target pressure is still less than the maximum target pressure the software next determines whether the actual pressure has equaled (risen to) the ascending target pressure 1812. If the actual pressure has attained the ascending target pressure, the software increments the variable target pressure by one interval 1814. Otherwise, it refrains from doing so until the actual pressure has equaled the ascending target pressure. If the variable target pressure has reached the maximum target pressure in the test of block 1810 the software sets the pressure direction to “decreasing” 1816 and the variable target pressure begins to move into the downward part of its oscillatory cycle.

The interval may be measured in mmHg or any other common unit of pressure measurement. The magnitude of the interval is preferably in the range of about 1 to 10 mmHg, according to the preference of the user.

If the actual pressure is decreasing in test 1808, a determination is then made as to whether the variable target pressure is still greater than the minimum target pressure 1818. If the variable target pressure is still greater than the minimum target pressure the software next determines whether the actual pressure has attained (fallen to) the descending target pressure 1820. If the actual pressure has equaled the descending target pressure the software decrements the variable target pressure by one interval 1822. Otherwise it refrains from doing so until the actual pressure has equaled the descending target pressure. If the variable target pressure has reached the minimum target pressure in the test of block 1818, the software sets the pressure direction to “increasing” 1824 and the variable target pressure begins to move into the upward part of its oscillatory cycle. This oscillatory process continues until the user de-selects the pulsing mode.

In the foregoing embodiments described with reference to Boynton, et al., the foam pad 400′ serves as the screen; the wound site 402′ serves as the tissue site; the wound drape 504 serves as the cover; the conduit 1402 serves as the liquid conduit; the canister 706 serves as the container; and the electrically powered pump 702 serves as the source of subatmospheric pressure. The appendage 508 serves as the connector interposed between the liquid conduit and the space defined by the cover to secure the liquid conduit to the cover. It is contemplated that the equipment may include wireless communication equipment to allow physicians to remotely access records of the conditions under which the patient has been treated.

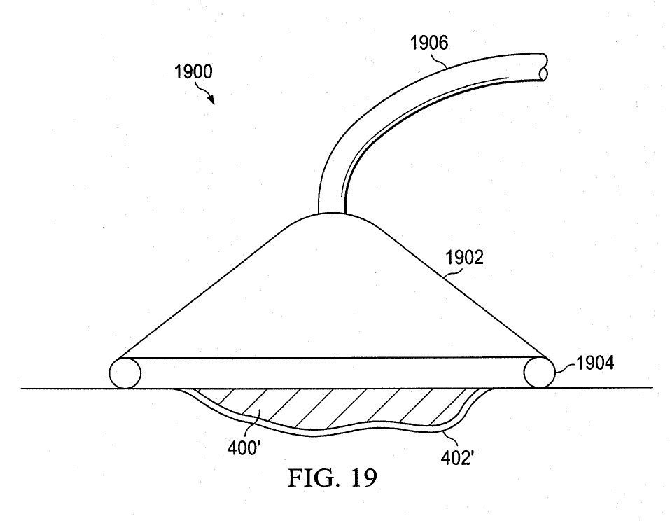

Alternate embodiments of the cover are contemplated including, but not limited to, semi-rigid covers that protect the tissue site 420′. FIG. 19 shows a cup-cuff cover 1900 comprising a semi-rigid cup 1902 and an inflatable cuff 1904. A conduit 1906 is connected to the source of subatmospheric pressure (not shown) and extends through a sealed aperture in the semi-rigid cup 1902. When inflated, the cuff 1904 conforms to the second region of tissue surrounding the tissue site 420′ and is held in place by application of the subatmospheric pressure within the space between the tissue and the cover.

The metallic properties of certain therapeutic or prophylactic agents, such as the antimicrobial silver, also lend themselves to metal-coating the dressing. Referring now to FIG. 20, a method 2000 for uniformly covering the foam dressing with the metallic silver coating is shown in the flow chart. First, stannous chloride and muriatic acid are combined to create a pre-metallizing solution, 2002. Any metal salt and/or acid capable of preparing the foam such that the metallic coating better adheres to the surface of the foam may be used in this embodiment. The solution is then placed in a first holding tank and agitated, 2004. The foam, which may comprise reticulated polyurethane die-cut foam, is placed in the first holding tank, 2006. The foam is then saturated with the pre-metallizing solution, which is accomplished through soaking or squeezing the foam, 2008. The foam is removed from the first holding tank and excess pre-metallizing solution is removed from the foam, 2010. Roller nips or similar devices may be utilized to control the amount of solution removed from the foam. A rinse solution is prepared in a second holding tank, 2012. The foam is immersed and thoroughly rinsed, 2014. The foam is removed from the second holding tank and excess rinse is removed from the foam, 2016.

Next, a silver oxide precipitate is combined in a solvent, such as ammonia, to create a silver-solvent complex, 2018. Any solvent capable of dissolving the metal and/or forming a metal-solvent complex may be used. The silver-solvent complex is then placed in a third holding tank and continuously agitated, 2020. The foam is placed in the third holding tank, 2022. The foam is then saturated with the silver-solvent complex, 2024.

Next, a surfactant is completely dissolved in deionized water and placed in a fourth holding tank, 2026. The foam is removed from the third holding tank and placed in the fourth holding tank, 2028. A reducing agent, such as formaldehyde, is added to the surfactant solution and agitated, and the foam is saturated in the solution, 2030. Any reducing agent that is capable of causing the metal to precipitate onto the substrate may be used in this embodiment. The reducing agent precipitates the silver onto the foam to form the metal-coated foam, 2032. The foam is removed from the fourth holding tank and excess solution is removed from the foam, 2034. A rinse solution is prepared in a fifth holding tank, 2036. The foam is immersed and thoroughly rinsed, 2038. The foam is removed from the fifth holding tank and excess rinse is removed from the foam, 2040.

Next, a mild caustic soda solution is prepared and placed in a sixth holding tank, 2042. The foam is immersed in the sixth holding tank and saturated in the caustic soda solution, 2044. The foam is removed from the sixth holding tank and excess caustic solution is removed from the foam, 2046. A rinse solution is prepared in a seventh holding tank, 2048. The foam is immersed and thoroughly rinsed, 2050. Next, the foam is removed from the seventh holding tank and excess rinse is removed from the foam, 2052. Optionally, the weight of the saturated foam, while still wet, may be calculated, 2054.

The foam is then placed in a convectional forced-air oven set to a predetermined temperature and time to completely dry the metal-coated foam, 2056. Alternatively, to verify the dry condition of the foam, the weight of the foam may be checked again, 2058. The foam is then packaged in a moisture vapor transmission rate pouch, if preferred, 2060. The foam is now ready for use on the tissue site, which may include without limitation, any site that may benefit from subatmospheric pressure tissue treatment, such as partial thickness burns, traumatic wounds, surgical wounds, dehisced wounds, diabetic wounds, pressure ulcers, leg ulcers, flaps and grafts.

It is understood that the foregoing coating-process steps, components, component proportions, the amount of time the substrate is immersed in the solutions, and the process for applying the solution to the substrate may vary to accommodate the substrate material and the agent to be coated on the substrate. Such variations are considered to be within the scope of this invention. Those skilled in the art can easily adapt the foregoing coating process for metal-coating other substrates, such as fiber or film, without undue experimentation.

A preferred embodiment uses a metallic coating process provided by Noble Fibers Technologies, Inc., of Clarks Summit, Pa., for producing the “V.A.C. GranuFoam® Silver” antimicrobial silver-coated foam dressing product commercialized by KCI USA, Inc. (and its affiliates) of San Antonio, Tex., for use in combination with their V.A.C.® subatmospheric pressure tissue treatment devices. Although portions of the metallic coating process used by Noble Fibers are proprietary and may not be publicly known, similar techniques will be known to those skilled in the art without undue experimentation.

The V.A.C. GranuFoam® Silver antimicrobial silver-coated foam dressing has achieved in-vitro efficacy on two common bacteria-staphylococcus aureus and pseudomonas aeruginosa, with a uniformly coated 99.9% pure silver metallic coating (4-12% silver by weight, though as little as 0.1% has shown to be at least partially effective). The coating is approximately 1-3 micrometers thick. The dressing maintains its effectiveness for at least 72 hours through a controlled and steady state release of ionic silver, providing over a 4 log reduction or about 99.99% of pathogenic bacteria have been eliminated between about 24 hours and about 72 hours. The coated dressing maintains the physical properties of the foam dressing substrate, which allows for direct and complete contact with the tissue site under application of the subatmospheric pressure.

An alternate embodiment includes uniformly coating a fiber substrate with a metallic agent, such as silver, wherein all fibers are circumferentially covered with the metallic coating. In this embodiment, the fiber is worked (woven, knitted, crocheted, felted, blown, etc.) to construct the dressing 400 subsequent the coating process. The uniform coating of the fiber substrate may be accomplished utilizing a metal-based coating process similar to the process 2000 of FIG. 20 without undue experimentation.

A similar process is used by Argentum Medical, LLC. of Chicago, Ill., for coating their “Silverlon®” antibacterial woven dressing product line. Although portions of the process are proprietary and may not be publicly known, similar techniques for metal-coating fiber, will be known to those skilled in the art.

While the foregoing description is exemplary of the preferred embodiments, those of ordinary skill in the relevant arts will recognize the many other alternatives, variations, alterations, modifications, substitutions and the like as are readily possible, especially in light of this description, the accompanying drawings and the claims drawn hereto. For example, those of ordinary skill in the art will recognize that while the preferred embodiment comprises grafting the desired agent onto the foam pad 11 of the subatmospheric pressure therapy system, those of ordinary skill in the art, with the benefit of this exemplary disclosure, will readily recognize many substantially equivalent modes for introduction of the desired agent. For example, in the case of a foam pad 11 that has not been predisposed with an agent or that has been predisposed with an agent which, over time, has subsequently been exhausted into the wound site 12, the desired agent may be injected with a needle and syringe, or the like, through the wound drape 13 and into the foam pad 11.

Further, it is contemplated that the components and additives for the polymer-based or metal-based coating solution may vary widely to accommodate the various substrate materials and agent(s) to be released. The coating can be formulated specific to coat thickness. It may be formulated to allow for various particle sizes. The coating may be formulated to provide various release kinetics, including but not limited to concentration, rate and the duration of agent release. For example, the release profile may be engineered such that release occurs in a matter of hours for up to several weeks. Concentration of delivery can be engineered to release from a low concentration of parts-per-billion (ppb) to several hundred parts-per-million (ppm) of agent within minutes. In the case where multiple agents are to be released, the coating may be formulated to provide scheduled and alternating agent releases.

Further, it is contemplated that the method of coating application or deposition may also vary widely, based on the various potential substrate materials and agent(s) to be released. The substrate material may vary beyond that set forth. Examples of the substrate useful in these embodiments include, but are not limited to foam, yarns, films, filaments, fibers, fabrics, filler materials, and a combination thereof that can be formed into the dressing 400.

It is also contemplated that the coating may incorporate single or multiple agents for release. Agents useful in these embodiments include, but are not limited to therapeutic and prophylactic agents, such as antimicrobial agents, enzymatic debriders, anesthetic agents, chemotherapeutic agents, indicating agents, and growth factors. Antimicrobial agents include, but are not limited to antibacterial agents, such as antibiotic and bacteriostatic agents. Useful indicating agents include, but are not limited to crystal violet, methylene blue, and similar agents known to cause a color change in tissue and/or body-liquid, for example, when in the presence of a bacterial agent, acidity, and alkalinity. Growth factors useful in embodiments discussed herein include, but are not limited to basic fibroblast growth factor, transforming growth factor, epidermal growth factor, platelet derived growth factor, insulin-like growth factor, keratinocyte growth factor, fibroblast growth factor, granulocyte macrophage colony stimulating factor, and granulocyte colony stimulating factor.

It is further contemplated that the screen 400 may comprise a plurality of portions, such as layers, only one of which comprises the uniformly covered substrate portion of the screen. In one embodiment, the screen 400 may be comprised of a lower uniformly covered substrate portion and an upper impermeable film portion of the screen, wherein the upper film portion of the screen may include an aperture or plurality of flow ports to provide fluid communication between the uniformly covered substrate portion of screen and the source of subatmospheric pressure. In an alternative embodiment, each of the plurality of portions of the screen may be comprised of substrate covered with different or alternating coatings for releasing a plurality of therapeutic or prophylactic agents to the tissue site 402.

We claim:

1. A reduced pressure therapy system comprising:
a foam pad having a plurality of passages to distribute a reduced pressure to a tissue, the foam pad having exterior surfaces, the foam pad further having interior surfaces along the plurality of passages;
a silver coating uniformly covering the exterior surfaces of the foam pad and the interior surfaces along the passages;
a drape adapted to be positioned over the foam pad to maintain a sealable space over the wound; and
a vacuum source in fluid communication with the sealable space to deliver the reduced pressure to the sealable space.

2. The system of claim 1, wherein the silver coating allows silver ions to be released to the tissue.

3. The system of claim 1, wherein the reduced pressure applied to the foam pad causes an increased area of contact between the foam pad and the tissue, thereby increasing exposure of the tissue to the silver coating.

4. The system of claim 3, wherein bacterial density at the tissue is reduced by causing the increased area of contact.

5. The system of claim 3, wherein the reduced pressure compresses the foam pad against the tissue to form the increased area of contact.

6. The system of claim 1, wherein bacterial density at the tissue is reduced by removal of body-liquid from the tissue into the passages and by the exposure of the body-liquid in the passage to the silver coating on the interior surfaces.

7. The system of claim 1, wherein the silver coating releases silver ions in an aqueous environment.

8. The system of claim 1, wherein the foam pad is an open-cell foam and the plurality of passages is interconnected pores within the foam pad.

9. The system of claim 1, wherein the silver coating includes at least one of silver hydrogel polymer, chitosan, and silver sodium aluminosilicate.

10. The system of claim 1, wherein the silver coating is one of polymer-based and metal-based.

11. The system of claim 1, wherein the foam pad is severable in any direction to expose a new surface uniformly coated by the silver coating.

12. The system of claim 1, wherein the silver coating has a thickness in a range of about 1 to 10 micrometers.

13. A reduced pressure apparatus comprising:
a foam pad having a plurality of passages to distribute a reduced pressure to a tissue, the foam pad having exterior surfaces, the foam pad further having interior surfaces along the plurality of passages;
a silver coating uniformly covering the exterior surfaces of the foam pad and the interior surfaces along the passages;
wherein the reduced pressure applied to the foam pad causes an increased area of contact between the foam pad and the tissue, thereby increasing exposure of the tissue to the silver coating and reducing bacterial density at the tissue;
wherein bacterial density at the tissue is further reduced by removal of body-liquid from the tissue into the passages and by the exposure of the body-liquid in the passages to the silver coating on the interior surfaces; and
wherein the foam pad is severable in any direction to expose a new surface uniformly coated by the silver coating.

14. The apparatus of claim 13, wherein the silver coating allows silver ions to be released to the tissue.

15. The apparatus of claim 13, wherein:
the reduced pressure compresses the foam pad against the tissue to form the increased area of contact; and
the reduced pressure removes the body-liquid from the tissue into the passages of the foam pad.

16. The apparatus of claim 13, wherein the silver coating releases silver ions in an aqueous environment.

17. The apparatus of claim 13, wherein the foam pad is an open-cell foam and the plurality of passages is interconnected pores within the foam pad.

18. The apparatus of claim 13, wherein the silver coating includes at least one of silver hydrogel polymer, chitosan, and silver sodium aluminosilicate.

19. The apparatus of claim 13, wherein the silver coating is one of polymer-based and metal-based.

20. The apparatus of claim 1, wherein the silver coating has a thickness in a range of about 1 to 10 micrometers.

21. A method for promoting wound healing comprising:
positioning a foam pad adjacent a wound, the foam pad having a plurality of passages to distribute a reduced pressure to the wound, the foam pad having exterior surfaces, the foam pad further having interior surfaces along the plurality of passages, the foam pad having a silver coating covering the exterior surfaces of the foam pad and the interior surfaces along the passages;
positioning a drape over the foam pad to create a sealable space;
delivering the reduced pressure to the sealable space;
promoting new tissue growth at the wound by exposing the wound to the reduced pressure; and
increasing an area of contact between the wound and the foam pad to increase the exposure of the wound to the silver coating.

22. The method of claim 21, wherein increasing an area of contact further comprises:
microdeforming the tissue under the influence of the reduced pressure.

23. The method of claim 21, wherein increasing an area of contact further comprises:
compressing the foam pad under the influence of the reduced pressure.

24. The method of claim 21 further comprising:
Reducing bacterial density at the wound by increasing exposure of tissue at the wound to the silver coating.

25. The method of claim 21 further comprising:
reducing bacterial density at the wound by removing body-liquid from the tissue into the passages and by exposing the body-liquid in the passages to the silver coating.
